(19) United States
(12) Patent Application Publication
(10) Pub. No.: US 2010/0198316 A1
(43) Pub. Date: Aug. 5, 2010

(54) Intracranial Red Light Treatment Device For Chronic Pain

(76) Inventors: Richard Toselli, Barrington, RI (US); Thomas M. DiMauro, Southborough, MA (US); Michael A. Fisher, Middleboro, MA (US); Sean Lilienfeld, Sharon, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/365,371

(22) Filed: Feb. 4, 2009

(51) Int. Cl. A61N 5/06

(52) U.S. Cl. 607/88

(57) ABSTRACT

Placement of a silicone tube in the cerebral aqueduct and the transmission of red light through it, resulting in the irradiation and consequent biostimulation of the adjacent periaqueductal gray, thereby causing the release of endorphins therefrom and pain relief.

### BACKGROUND OF THE INVENTION

The leading cause of lower back pain arises from rupture or degeneration of lumbar intervertebral discs. Pain in the lower extremities is caused by the compression of spinal nerve roots by a bulging disc, while lower back pain is caused by collapse of the disc and by the adverse effects of articulation weight through a damaged, unstable vertebral joint. One proposed method of managing these problems is to remove the problematic disc and replace it with a porous device that restores disc height and allows for bone growth therethrough for the fusion of the adjacent vertebrae. These devices are commonly called “fusion devices”. Although the use of fusion devices to treat back pain has become increasingly popular, there remains a significant proportion of patients who undergo this surgery and yet still experience chronic back pain. This phenomenon is called “failed back syndrome”.

Deep Brain Stimulators (DBS) have been used to treat chronic pain, including failed back syndrome. In this treatment, electrodes are often placed in the periaqueductal grey (PAG) region of the brain. The periaqueductal gray (PAG) has a very important antinociceptive function, and its stimulation decreases pain. When the DBS electrodes are activated, the periaqueductal grey is stimulated and releases pain-reducing endorphins. In one study examining the efficacy of DBS in relieving chronic pain, 47% of the patients treated with DBS electrodes suffered from failed back syndrome. Therefore, it appears that stimulation of the PAG can provide significant pain relief for patients suffering from failed back syndrome.

Although DBS has had some success as a medical implant, this mode of treatment also has some drawbacks. For example, it appears that scar tissue forms around the electrodes, causing their failure in many cases after about two years. In addition, Since the patient's anatomy controls the flow of electrical current, it is difficult to control the location and dose of the current. Moreover, it is believed that electricity jolts or provokes cellular response,rather than enabling or eliciting response. Accordingly, it is not clear whether such jolting will yield adurable effect or merely tire the provoked cells.

US Patent Publication No. 2006/0155348 (deCharms) teaches irradiation of a number of brain regions, including the PAG, with various wavelengths of light. However, deCharms teaches that the irradiation should be of a sufficiently large scale as to cause electrical current to flow through the irradiated region. The level of irradiation required to cause such a current greatly exceeds the level commonly used in low level laser therapy (LLLT).

### SUMMARY OF THE INVENTION

It has been reported in the literature that low level irradiation of tissue with red light stimulates the release of pain-reducing endorphins from the irradiated cells. For example, Laakso, Photomed Laser Surg. February 2005;23(1):32-5 induced inflammation in the hind-paws of Wistar rats. Two groups of rats then received 780-nm laser therapy at one of two doses (2.5 J/cm² and 1 J/cm²). Scores of nociceptive threshold were recorded using paw pressure and paw thermal threshold measures. Laakso found that a dose of 2.5 J/cm² provided a statistically significant effect on paw pressure threshold (p<0.029) compared to controls. Laakso further found normal beta-endorphin containing lymphocytes in control inflamed paws but no beta-endorphin containing lymphocytes in rats that received laser at 2.5 J/cm². Without wishing to be tied to a theory, it is believed that these results appear to show the release of endorphins from the lymphocytes of the irradiated rats. Lastly, Zalewska-Kaszubska, Lasers Med Sci. 2004;19(2):100-4, reported treating patients with 20 consecutive daily helium-neon laser neck biostimulations and 10 auricular acupuncture treatments with argon laser (every 2nd day), and finding that the beta-endorphin plasma concentration in those patients was increased.

Therefore, it is believed that low level red light irradiation of the PAG should also cause release of pain-reducing endorphins from the PAG, thereby affording pain relief to the patient suffering from chronic pain.

In the present invention, the PAG is locally stimulated through low level laser therapy to elicit pain relief. In some embodiments, the placement of a light-diffusing tube in the cerebral aqueduct and the transmission of red light through it will result in the irradiation of the adjacent PAG, thereby causing the release of endorphins and pain relief.

Therefore, in accordance with the present invention, there is provided a method of treating a patient having chronic pain, comprising the steps of:
a) providing a optical wave guide having a proximal end portion and a distal end portion having a translucent light diffuser (preferably, in the form of a tube) attached thereto;
b) implanting the translucent light diffuser into the patient's cerebral aqueduct, and
c) delivering light through the optical wave guide to irradiate at least a portion of a periaqueductal gray with an effective amount of light to cause release of endorphins from the periaqueductal gray.

### DESCRIPTION OF THE FIGURES

FIG. 1 discloses a cross-section of the brain in which the cerebral aqueduct connects the third ventricle with the fourth ventricle.

FIG. 2 discloses a device of the present invention having a translucent tube at the distal end of the device.

FIG. 3a discloses a cross-section of the first translucent tube and the distal end of the optical wave guide implanted in the cerebral aqueduct.

FIG. 3b discloses a cross-section of the longer translucent tube and the distal end of the optical wave guide implanted in the cerebral aqueduct.

FIGS. 3c and 3d disclose cross-sections of a light diffuser comprising a central element and a plurality of radially-extending standoffs.

FIG. 4 is a cross-section of an LED implant of the present invention implanted within the brain of a patient having chronic pain.

FIG. 5 is a cross-section of an implant of the present invention having a optical wave guide and implanted within the brain of a patient having chronic pain.

FIGS. 6A-6B are cross-sections of a optical wave guide implant of the present invention implanted within the brain of a patient having chronic pain.

FIG. 6C is a cross-section of a optical wave guide implant of the present invention.

FIG. 6D discloses a convex lens added to the red light collector situated in the skull.

FIG. 6e discloses a transparent replacement material between the implant and the epidermis.

FIG. 7a is a cross-section of an implanted optical wave guide implant irradiated by a light source.

FIG. 7b is a cross-section of an implanted optical wave guide implant having a gasket irradiated by a light source.

FIG. 8 is a cross-section of an Rf source energized an LED implant of the present invention.

FIG. 9 is a cross-section of an LED implant of the present invention.

FIG. 10 is a schematic of electronics associated with an LED implant of the present invention.

FIG. 11 is a cross-section of a toothed LED implant of the present invention implanted within the brain of a patient having chronic pain.

### DETAILED DESCRIPTION

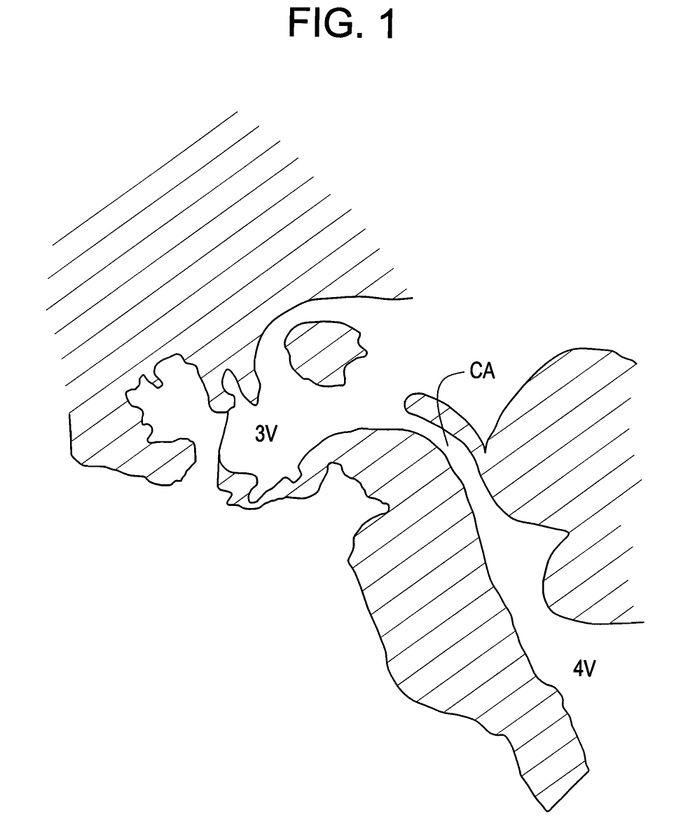

Now referring to FIG. 1, there is provided a cross-section of the brain in which the cerebral aqueduct CA connects the third ventricle 3V with the fourth ventricle 4V.

In one preferred embodiment of the present invention, the distal end of the optical wave guide is attached to a translucent light diffuser, which is often in the form of a tube. The light diffuser is placed in the cerebral aqueduct and acts not only as a light delivery device to the PAG (which surrounds the cerebral aqueduct), but also as an anchor within the compliant cerebral aqueduct that holds the device in place.

The literature has repeatedly reported the successful placement of stents in the cerebral aqueduct as a method of managing blockage of the cerebral aqueduct or fourth ventricle. See, for example, Shin, J. Neurosurg., June 2000 92(6) 1036-9; Cinalli, J. Neurosurg., January 2006, 104(1 Supp.) 21-7; Sagan, J. Neurosurg. (4 Supp pediatrics) 105: 275-280, 2006; Schroeder, Operative Neurosurgery, 1, 60, February 2007 ONS-44-52. Therefore, placement of the translucent light diffuser in the form of a tube of the present invention in the cerebral aqueduct is a procedure that should be well within the expertise of the neurosurgeon.

The placement of the translucent light diffuser essentially adjacent the PAG has special advantage in that there is no intervening brain tissue between the tube and the PAG. Therefore, there is no need to estimate how much light would be attenuated or diffracted or reflected by the intervening tissue as the light proceeds from the translucent light diffuser through that intervening tissue and on to the target tissue. Thus, the amount of light that exits the light diffuser is essentially equal to the amount of light that irradiates the PAG, and so energy fluency at the PAG can be reasonably estimated by using the outer surface area of the light diffuser. Since there is no need for overirradiating any intervening tissue in order to obtain sufficient fluency at the PAG, there is no danger of overheating or destimulating any intervening brain tissue.

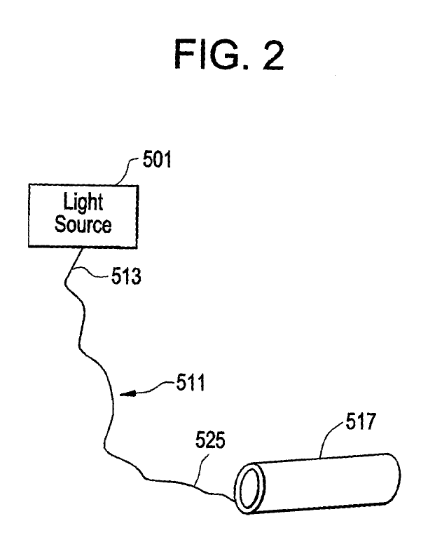

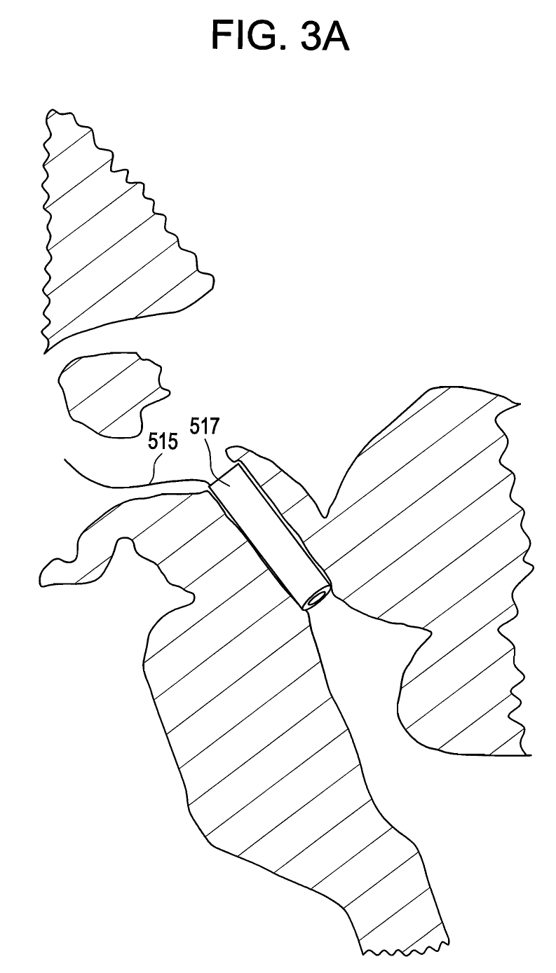

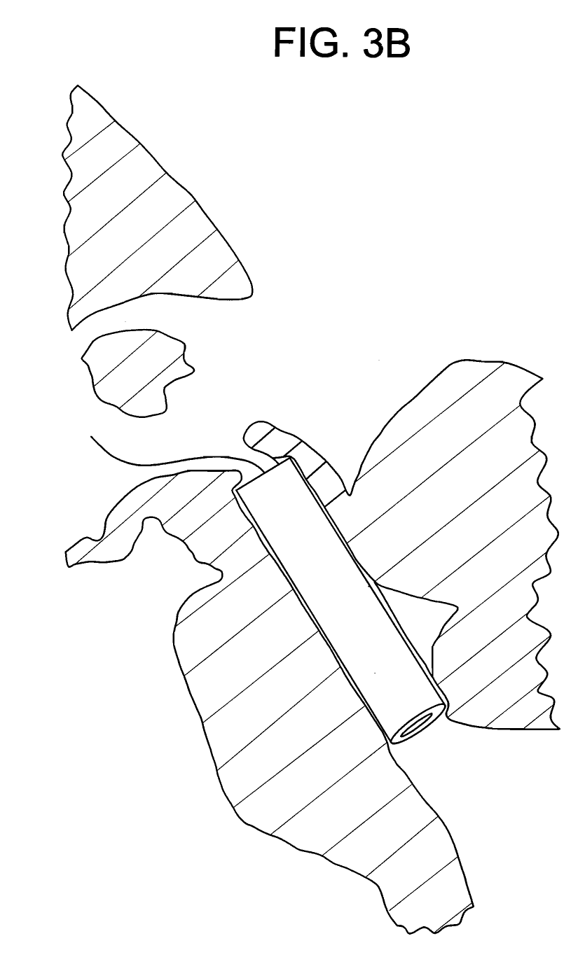

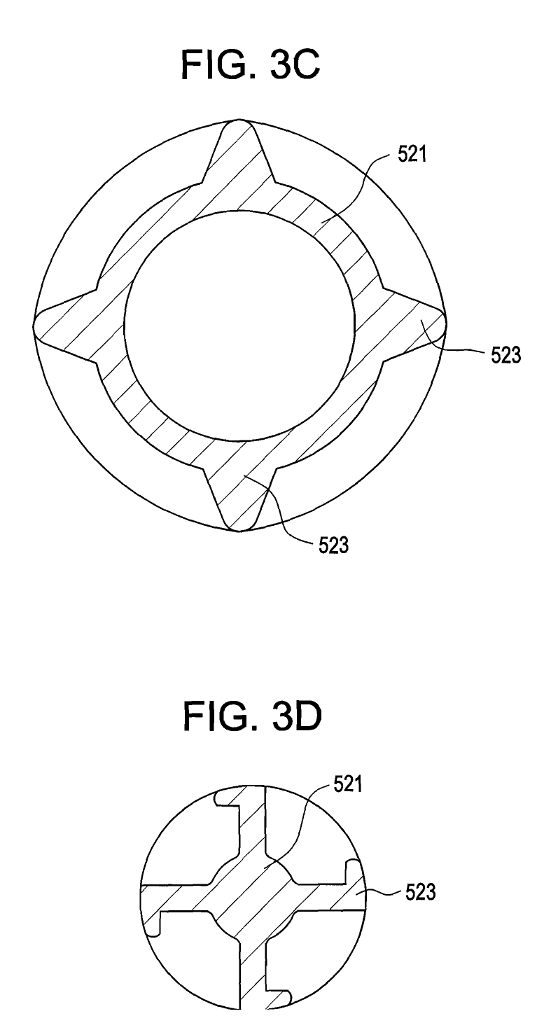

Therefore, in accordance with the present invention, and now referring to FIG. 2, there is provided an intracranial light delivery system, comprising:
a) a light source 501,
b) a optical wave guide 511 having a proximal end 513 connected to the light source and a distal end 515, and
c) a translucent light diffuser 517 connected to the distal end of the optical wave guide.
   In general, the larger the diameter of the translucent light diffuser, the more snug will be its fit within the cerebral aqueduct (which typically has a relaxed diameter of about 1 mm in the average adult). Because the cerebral aqueduct is endowed with a compliance, it can accommodate instruments up to 4 mm in width (Longatti, Neurosurg. Focus 19(6) E12, 2005. FIG. 3a discloses the translucent light diffusing tube 517 and the distal end 515 of the optical wave guide implanted in the compliant cerebral aqueduct. Therefore, in some embodiments, the diameter of the translucent light diffuser is at least 2 times the diameter of the relaxed cerebral aqueduct, and preferably at least about 3 times the diameter of the relaxed cerebral aqueduct. In general, the compliance of the cerebral aqueduct is such that it can expand to a diameter of about 4 mm in the typical adult. Therefore, in some embodiments, the diameter of the translucent light diffuser is about 4 times the diameter of the relaxed cerebral aqueduct. In this embodiment, the snugness of the fit within the cerebral aqueduct is maximized.

Because of the ability of the cerebral aqueduct to accommodate large changes in diameter, it might be possible to directly illuminate the PAG by implanting the light source directly in the cerebral aqueduct without any intervening optical wave guides (see LED chain image copied from FIG. 3A depicting an implanted light source where the LED's are located in beads that are placed inside the cerebral aqueduct and the incoming signal is an electrical signal from a superficial/distal power supply and controller instead of an optical fiber).

In general, the longer the length of the translucent light diffuser, the more reliable will be its fit within the cerebral aqueduct (which has a length of about X cm in the typical adult). Therefore, in some embodiments, the length of the translucent light diffuser is at least 25% of the length of the cerebral aqueduct, preferably at least about 50% of the length of the cerebral aqueduct, and more preferably at least about 75% of the length of the cerebral aqueduct. However, in some embodiments, the length of the translucent light diffuser is no more than 90% of the length of the cerebral aqueduct. In this condition, the ends of the cerebral aqueduct will form front and back lips that function as shoulders to keep the light diffuser in place and resist its migration. FIG. 3b discloses a longer translucent light diffuser and the distal end of the optical wave guide implanted in the compliant cerebral aqueduct, wherein the tube spans nearly the entire cerebral aqueduct.

The translucent light diffuser can include a rim, lips, ribs, threads, flair, stand-offs, folds, hooks, posts, trumpet end flair, loops, or helix to prevent migration of the device. Additionally, several of these embodiments would enable increased local tissue diffusion at the light diffuser-tissue interface thereby mitigating any metabolic issues resulting from device placement.

In some embodiments, the translucent light diffuser comprises silicone. Silicone tubes are currently used as ventricular catheters in the treatment of hydrocephalus. In addition, the literature has reported the use of silicone tubes as lumen-opening stents in general surgery. See, for example, Westaby, British Journal Surgery. May 1983;70(5):259-60; Roh, Dsphagia. April 2006;21(2):112-5. In addition, silicone is fairly translucent to red light. In some embodiments, the translucent light diffuser consists essentially of silicone.

Additional silicone embodiments can include hollow channels with reflective internal/external coatings.

In some embodiments, and now referring to FIGS. 3a and 3b, the light diffuser at the distal end of the implant comprises a tube shape. In this configuration, the light diffuser can act as a stent within the cerebral aqueduct, keeping itself in place while providing therapeutic light energy to the PAG.

Although the tube shape beneficially diffuses light to the entirety of the aqueduct perimeter, it may also restrict fluid flow from the aqueduct to the PAG. Therefore, and now referring to FIG. 3c, in some embodiments, the light diffuser comprises a central element 521 and a plurality of radially extending standoffs 523. The standoffs provide space between the central element of the light diffuser and the PAG, thereby allowing CSF within the aqueduct to reach the PAG. FIG. 3c demonstrates how the standoffs act to center the central element within the aqueduct. Providing standoffs also reduces the contact area of the light diffuser with sensitive brain tissue, and allows surface diffusion between the CSF and the PAG tissue. Because of the relatively quiescent nature of brain tissue, there is a relatively low likelihood of tissue ingrowth and adhesion. FIG. 3d also demonstrates how the standoffs contour to the local geometry, thereby reducing the likelihood of implant migration.

In some embodiments, antibiotics such as BACTISEAL™, are impregnated into the silicone tube. Additionally, silver coatings can be used to increase surface reflectance and impart anti-biotic and anti-bacterial colonization attributes to the part (SilvaGard™ silver nano particles by AcryMed of Beaverton, Oreg.).

In preferred embodiments, the translucent light diffuser possesses features that increase the radial transmission of light through its outer surface. In some embodiments, diffractive elements, such as metallic particles, are embedded within the translucent tube in order to diffract light that is traveling down the length of the tube to cause that light to exit the tube in a radial direction. In other embodiments, the outer surface of the tube is etched in order to diffract light that is traveling down the length of the tube to cause that light to exit the tube in a radial direction. In some embodiments, the distal end of the tube is coated with a reflective coating to deflect axially-traveling light back into the tube. In some embodiments, the inner surface of the tube is coated with a reflective coating to deflect light back into the tube. In further embodiments, the tube is allowed to “leak” light through the internally reflective coating to achieve radial illumination. Similarly, the external contours of the tube wave guide can be designed to allow radial light diffusion (sinusoidal or crenulated surfaces will leak more light than smooth surfaces).

In some embodiments, the outer surface of the translucent tube is coated with an adhesive in order to insure the retention of the tube within the cerebral aqueduct. One adhesive, polyethylenimine, has been tested as an adhesive for bonding electrodes to neurons. He, Biomaterials 26 (2005) 2983-2990. It appears to be a non-resorbing adhesive and promotes neuron attachment to itself. However, test data is limited to about 15 days. Sutures, staples, stents, lock & key, in situ curing/stiffening of the device to contour to the unique shape of the aqueduct.

In some embodiments, an implanted optical wave guide is used to deliver photonic energy from the proximal light collector to a location within the brain. The optical wave guide can be embodied as an optical fiber, internally-reflective tube (or “light pipe”), diffusion/diffraction surface(s), optical lens and mirror system, etc. or a combination of these elements.

In some embodiments, the optical wave guide is a light pipe. In one embodiment, the light pipe is a truncated form of the Flexible Light Pipe FLP 5 Series, marketed by Bivar Inc., which is a flexible light pipe that is 12 inches long and 2 mm in diameter, and has an outer tubing of fluorinated polymer TFE.

In some embodiments, the optical wave guide is a coiled sheet or convoluted surface that guides optical energy (light) from a source, through the light diffuser to a final target (in this case, a tissue or anatomical region of the brain, PAG). The benefit of a hollow optical wave guide is the decreased amount of light energy being absorbed by the material conduit. This benefit is mitigated by optical inefficiencies due to imperfect reflectance, but light attenuation by absorption will be greatly reduced in a hollow internally reflecting optical wave guide.

Silicone might also be used as the core and/or cladding of an optical fiber as long as the materials have different optical refractive indices. Those practiced in the art will appreciate how to manufacture silicone cores with silicone cladding.

Alternatively, a traditional optical material like glass or clear acrylic can be used as the optical wave guide core with silicone cladding that also serves as a biological boundary to impart overall device biocompatibility.

Because the delivery and placement of the light diffuser takes places entirely within the ventricular system of the brain, such delivery and placement may be performed endoscopically. The endoscopic delivery and placement of this system represents a significant advantage over the conventional stereotactically-guided placement of medical devices in the brain. First, whereas stereotactically guided systems require the use of expensive and complicated hardware, endoscopic placement of a tube within the cerebral aqueduct is relatively straightforward and can be performed without expensive and time-consuming support equipment. Second, stereotactically guided systems typically require blunt invasion of the brain parenchyma and its related vasculature, and so generate a risk of producing neural deficits and hemorrhage. For example, Kleiner-Fisman, Mov. Disord., Jun. 21, 2006, Suppl. 14 S290-304 reports a 3.9% hemorrhage rate for Parkinson's patients receiving deep brain stimulation implants. In contrast, endoscopic placement of a stent in the cerebral aqueduct does not produce any injury to the brain tissue or its related vasculature whatsoever, and so therefore should completely eliminate the risk of hemorrhage.

In sum, endoscopically accessing the ventricular system is much less complicated than placing a catheter directly into the brain parenchyme. Endoscopic access could be performed by most neurosurgeons and so there would be no need to require stereotactic-trained surgeons or stereotactic/navigation equipment. Most neurosurgeons are capable of and would be comfortable placing a tube into the lateral ventricle and driving that catheter into the floor of the third ventricle endoscopically and then into the cerebral aqueduct. Anatomic landmarks would facilitate its placement and this would obviate the need for complex stereotactic localizing techniques. It would a simpler procedure for patients and could be performed by most neurosurgeons.

Therefore, in accordance with the present invention, there is provided a method of treating a patient having chronic pain, comprising the steps of:
a) providing an optical wave guide having a proximal end portion and a distal end portion having a translucent tube attached thereto;
b) endoscopically implanting the tube into the patient's cerebral aqueduct, and
c) delivering light through the optical wave guide to irradiate at least a portion of a periaqueductal grey with an effective amount of light.

In some embodiments using endoscopic placement, a modified procedure of Farin, “Endoscopic Third Ventriculostomy” J. Clin. Neurosci. August;13(7):2006,763-70 is used. In particular, a burr hole is made through the skull at the intersection of the coronal suture and the midpupillary line, approximately 2-3 cm lateral to the midline. The endoscope trajectory is aimed medially toward the medial canthus of the ipsilateral eye and toward the contralateral external auditory meatus in the anterior/posterior plane. This approach leads to the foramen of Monro and floor of the third ventricle. The lateral aspect of the anterior fontanelle is targeted. The dura is opened. The lateral ventricle is tapped using a peel-away sheath with ventricular introducer. The sheath is secured in place to the scalp. A rigid neuroendoscope is then inserted into the lateral ventricle, and the choroid plexus and septal and thalamostriate veins are identified in order to locate the foramen of Monro. The endoscope is advanced into the third ventricle. The mamillary bodies are some of the more posterior landmarks of the third ventricle; moving anteriorly, the basilar artery, dorsum sellae and infundibular recess may be obvious based on the degree of attenuation of the ventricular floor. The endoscope is then moved farther posteriorly to the posterior end of the third ventricle to reach the mouth of the cerebral aqueduct. The endoscope is then inserted into the cerebral aqueduct, wherein it deposits the translucent tube portion of the device.

Without wishing to be tied to a theory, it is believed that the therapeutic effects of red light described above may be due to an increase in ATP production in the irradiated neurons. It is believed that irradiating neurons in the brain with red light will likely increase ATP production from those neurons. Mochizuki-Oda, Neurosci. Lett. 323 (2002) 208-210, examined the effect of red light on energy metabolism of the rat brain and found that irradiating neurons with 4.8 W/cm² of 830 nm red light increased ATP production in those neurons by about 19%.

Without wishing to be tied to a theory, it is further believed that the irradiation-induced increase in ATP production in neuronal cell may be due to an upregulation of cytochrome oxidase activity in those cells. Cytochrome oxidase (also known as complex IV) is a major photoacceptor in the human brain. According to Wong-Riley, Neuroreport, 12:3033-3037, 2001, in vivo, light close to and in the near-infrared range is primarily absorbed by only two compounds in the mammalian brain, cytochrome oxidase and hemoglobin. Cytochrome oxidase is an important energy-generating enzyme critical for the proper functioning of neurons. The level of energy metabolism in neurons is closely coupled to their functional ability, and cytochrome oxidase has proven to be a sensitive and reliable marker of neuronal activity.

By increasing the energetic activity of cytochrome oxidase, the energy level associated with neuronal metabolism may be beneficially increased.

Preferably, the red light of the present invention has a wavelength of between about 600 nm and about 1000 nm. In some embodiments, the wavelength of light is between 800 and 900 nm, more preferably between 825 nm and 835 nm. In this range, red light has not only a large penetration depth (thereby facilitating its transfer to the optical wave guideand SN), but Wong-Riley reports that cytochrome oxidase activity is significantly increased at 830 nm, and Mochizuki-Oda reported increased ATP production via a 830 mn laser.

In some embodiments, the wavelength of light is between 600 and 700 nm, and preferably is 670 nm. In this range, Wong-Riley reports that cytochrome oxidase activity was significantly increased at 670 nm. Wollman reports neuroregenerative effects with a 632 nm He—Ne laser.

In some embodiments, the light source is situated to irradiate adjacent tissue with between about 0.01 J/cm² and 20 J/cm² energy. Without wishing to be tied to a theory, it is believed that light transmission in this energy range will be sufficient to increase the activity of the cytochrome oxidase around and in the target tissue. In some embodiments, the light source is situated to irradiate adjacent tissue with between about 0.05 J/cm² and 20 J/cm² energy, more preferably between about 2 J/cm² and 10 J/cm² energy.

In accordance with US Patent Publication 2004-0215293 (Eells), LLLT suitable for the neuronal therapy of the present invention preferably has a wavelength between 630-1000 nm and power intensity between 25-50 mW/cm² for a time of 1-3 minutes (equivalent to an energy density of 2-10 J/cm²). Eells teaches that prior studies have suggested that biostimulation occurs at energy densities between 0.5 and 20 J/cm², whereas energy densities above 20 J/cm² may exert bioinihibitory effects. Preferable energy density of the present invention is between 0.5-20 J/cm², most preferably between 2-10 J/cm². In summary, a preferred form of the present invention uses red and near infrared wavelengths of 630-1000, most preferably, 670-900 nm (bandwidth of 25-35 nm) with an energy density fluence of 0.5-20 J/cm², most preferably 2-10 J/cm², to produce photobiomodulation. This is accomplished by applying a target dose of 10-90 mW/cm², preferably 25-50 mW/cm² LED-generated light for the time required to produce that energy density.

In general, the amount of light irradiating the PAG should be less than about 20 J/cm². Above this 20 J/cm² amount, it is believed that LLLT works to inhibit biometabolism. For example, Byrnes, Lasers Surg. Med., 9999:1-15(2005) found high laser dosages to be inhibitory and cited another reference (Tuner, “Laser Therapy: Clinical Practice and Scientific Background”. Tallinn, Estonia: Prima Books AB, 2002) for the proposition that doages greater than 10 J/cm² are inhibitory.

In some embodiments, the light source is situated to produce an energy intensity of between 0.1 watts/cm² and 10 watts/cm². In some embodiments, the light source is situated to produce about 10-90 milliwatt/cm², and preferably 7-25 milliwatt/cm².

Wong-Riley Neuroreport 12(14) 2001:3033-3037 reports that a mere 80 second dose of red light irradiation of neuron provided sustained levels of cytochrome oxidase activity in those neurons over a 24 hour period. Wong-Riley hypothesizes that this phenomenon occurs because “a cascade of events must have been initiated by the high initial absorption of light by the enzyme”.

Therefore, in some embodiments of the present invention, the therapeutic dose of red light is provided on approximately a daily basis, preferably no more than 3 times a day, more preferably no more than twice a day, more preferably once a day.

In some embodiments, the red light irradiation is delivered in a continuous manner. In others, the red light irradiation is pulsed in order to reduce the heat associated with the irradiation. In some embodiments, red light is combined with polychrome visible or white light

Thus, there may be a substantial benefit to providing a local radiation of the PAG with red laser light. The red light can be administered in a number of ways:
1) By implanting near the skull an implant having a red light LED, an antenna and a thin optical wave guide terminating at the PAG, and telemetrically powering the LED via an external antenna to deliver red light through the optical wave guideto the PAG.
2) By placing a optical wave guide having a proximal light collector at the interior rim of the skull and running it to the PAG, and then irradiating the proximal end via an external red light source. Red light can penetrate tissue up to about one cm, so it might be able to cross the skull and be collected by the collector.
3) By implanting a red light LED in the skull, and powering the LED via an internal battery.
   In each case, there is produced an effective amount of local red or infrared irradiation around the PAG. This light would then increase local ATP production, thereby increasing the metabolism in the PAG.

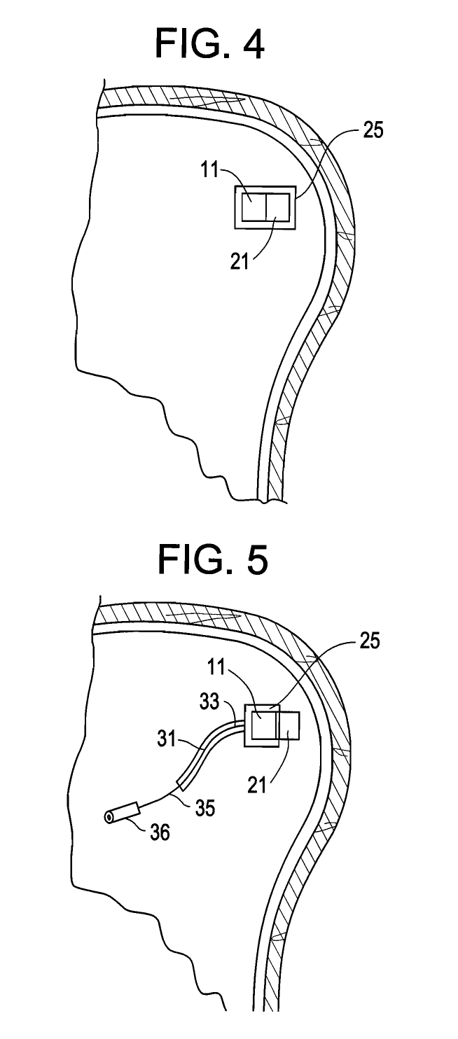

Now referring to FIG. 4, there is provided an implant for treating pain comprising:
a) a Red Light emitting diode (LED) 11, and
b) an antenna 21 in electrical connection with the LED.

In use, the surgeon implants the device into the brain of the patient so that the device is adjacent to a portion of the PAG. The Red light produced by the implant will then irradiate that portion of the PAG.

In order to protect the active elements of the device from cerebrospinal fluid (“CSF”), in some embodiments, and again referring to FIG. 4, the Red light LED is encased in a casing 25. This casing both protects the LED components from the CSF, and also prevents the LED components from elicting undesirable immune reactions. In some embodiments, the casing is made of a Red light transparent material. The Red light transparent material may be placed adjacent the LED component so that Red Light may be easily transmitted therethrough. In some embodiments, the transparent casing is selected from the group consisting of silica, alumina and sapphire. In some embodiments, the light transmissible material is selected from the group consisting of a ceramic and a polymer. Suitable red light-transmissible ceramics include alumina, silica, CaF, titania and single crystal-sapphire. Suitable light transmissible polymers are preferably selected from the group consisting of polypropylene and polyesters.

In some embodiments, it may be desirable to locate the light emitting portion of the implant at a location separate from the LED, and provide a light communication means between the two sites. The light communication means may include any of a optical wave guide, a wave guide, a hollow tube, a liquid filled tube, and a light pipe.

Now referring to FIG. 5, there is provided an implant 1 for treating chronic pain comprising:
a) a Red Light emitting diode (LED) 11,
b) an antenna 21 in electrical connection with the LED, and
c) a optical wave guide 31 adapted to transmit Red light, the guide having a proximal end 33 connected to the LED an and a distal end portion 35, and
d) a translucent tube 36 connected to the distal end portion of the guide.
   Such a configuration would allow the distal end of the optical wave guide (and translucent tube) to be located deep within the patient's brain near the PAG and yet have the light source and associated components located near or in the skull in a less sensitive region. This configuration allows easier access to the light/controller should the need arise for service or maintenance, and also allow for more efficient transdermal energy transmission. The light source/controller implanted near the patient's skull could also be a simple, hollow chamber made to facilitate the percutaneous access described above. The advantages and benefits of this system include further removal from the deep site of the functional implant, thereby reducing risk of contamination of the deeper site by percutaneous access, and easier precutaneous access by being closer to the skin surface and having a larger surface area or target to access with the needle.

In use, the surgeon implants the device into the brain of the patient so that the antenna is adjacent the cranium bone and the distal end of the optical wave guide is adjacent to the PAG region of the brain.

In some embodiments, the proximal end portion of the optical wave guide is provided with a cladding layer 41 of reflective material to insure that Red light does not escape the guide into untargeted regions of brain tissue.

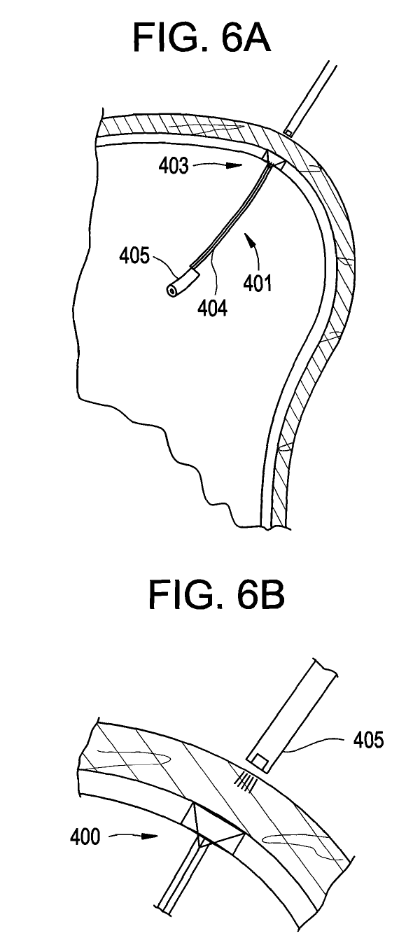

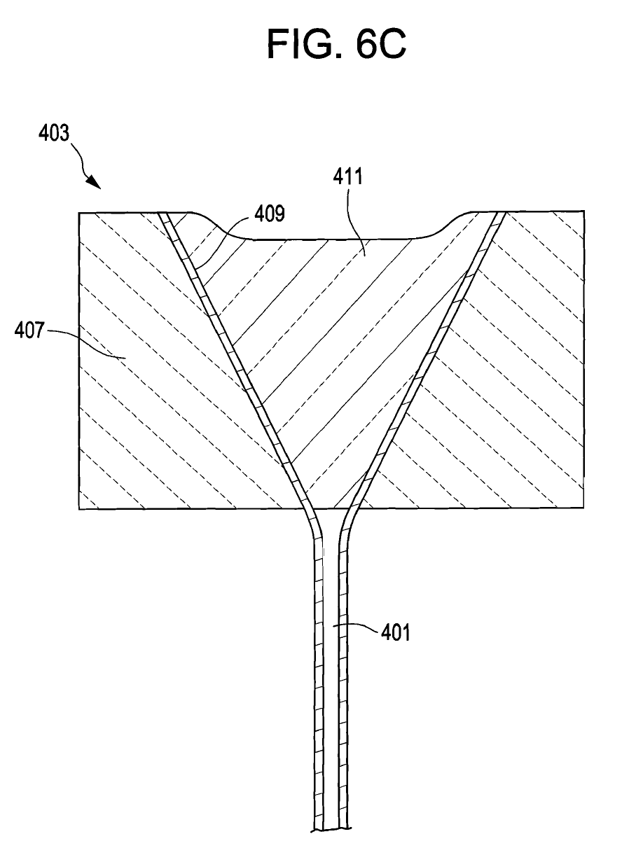

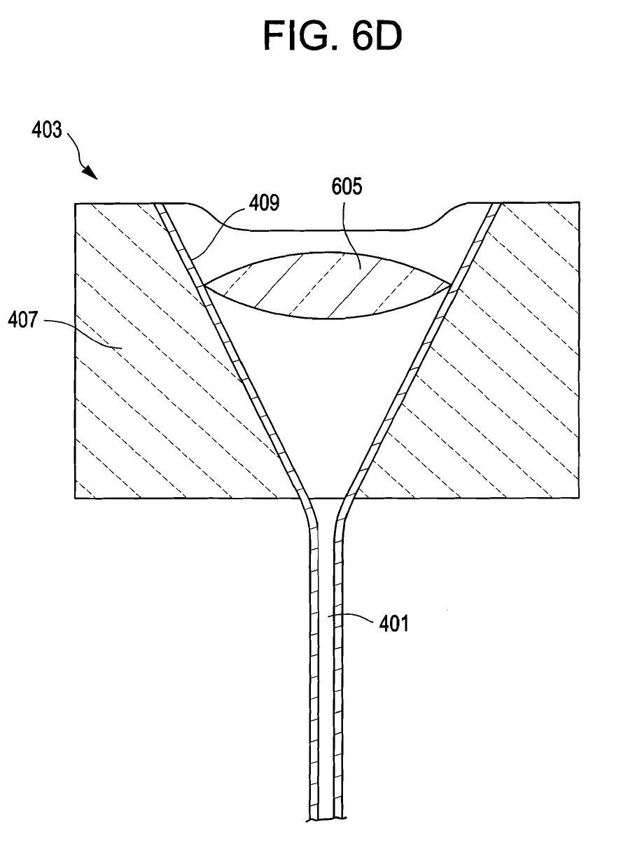

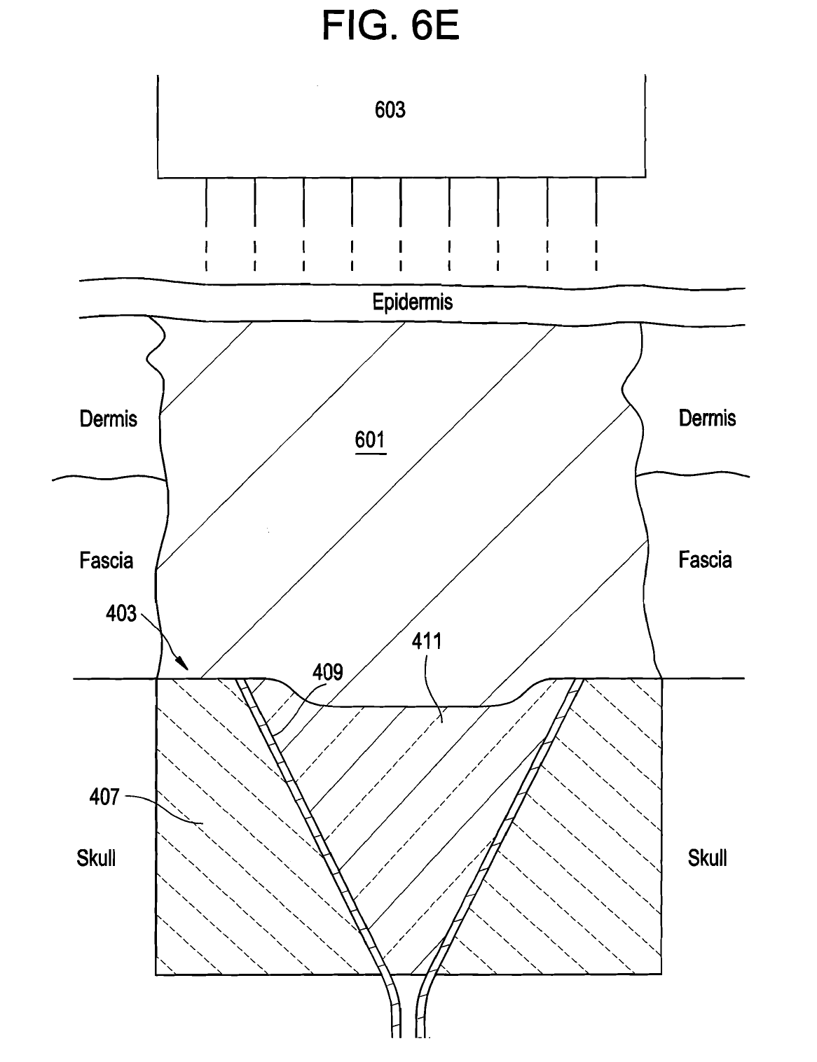

Because long wavelength red light can penetrate up to many centimeters, it might be advantageous to transcutaneously deliver the light the fiber optic. Now referring to FIGS. 6a-6c, in one embodiment, a optical wave guide 401 having a proximal light collector 403 is placed at the interior rim of the skull. The distal end portion 404 of the guide is connected to the tube 405 and is placed in the cerebral aqueduct. Red light can then be delivered transcutaneously from a probe 415 to the collector 403, which will then transport the light through the guide and the tube to the PAG.

In some embodiments, as in FIG. 6c, the collector 403 has a porous osteoconductive collar 407 for intergrating with the bone in the skull. The collector may comprise a funnel-shaped mirror 409 (made of titanium) that connects to the optical wave guide 401 and is filed with a red light-transparent material 411 such as silica.

To enhance the propagation of light emitted from the end of the fiber, a lens could be placed at the distal end of the fiber to spread the light, or a diffuser such as a small sheet or plate of optical material could be used to create more surface area. Alternatively, one could create a series of lateral diffusers, such as grooves or ridges, along the distal portion of end of the fiber to spread light out from 360 degrees perpendicular to the axis of the fiber, as well as emanating directly out from the end of the fiber.

In some embodiments using the transcutaneous delivery of red light, the receiving portion of the device is fitted with a lens to focus the light into the proximal end portion of the optical wave guide. In particular, and now referring to FIG. 6d, a convex lens 605 is added to the red light collector situated in the skull. The lens would be able to focus the incoming light that passes through the scalp to a point corresponding to the mouth of the fiber optic. This focusing would mitigate any problems associated with losing light as the light transitions from the collector to the fiber optic. It would also allow the user to irradiate with a low power light over a broader scalp area and still obtain a concentrated beam in the fiber optic. This would mitigate any issues associated with overirradiating the scalp tissue.

In some embodiments, red light is provided to the brain via delivery through the scalp. These are highly preferred embodiments because they are non-invasive. However, it is recognized that there may be some light loss associated with this mode of delivery. The literature reports that while the epidermis portion of the scalp is essentially transparent to light, the dermis and fascia portions of the scalp are light-attenuating and light-diffracting due to the presence of blood vesssels and fat in these layers.

Therefore, in some embodiments of the present invention, a core is taken of at least a portion of the subepidermal tissue residing between the skull and the epidermis, and that cored volume of tissue is replaced with a transparent material. Preferably, a core is taken of substantially all of the subepidermal tissue between the skull and the epidermis, and that tissue is replaced with a transparent material. In this condition, light delivered from an ex vivo source will need only to pass through the transparent epidermis and then the transparent replacement material in order to enter the light collector portion of the implant. This should greatly attenuate any light loss associated with transcutaneous light delivery.

Preferably, the transparent material is a gel. When it is in a gel state, the transparent material has the ability to smoothly and evenly respond to external pressures on the epidermis, thereby mitigating concerns of the transparent replacement material breaching the epidermis due to external pressures on the epidermis.

The literature reports that the thickness of the epidermis in the scalp is about 2-3 mm. Bukhari, Ann. Saud. Med. 24(6) November-December 2004 484-485. It is believed that such a thickness would be adequate to safely cover and house the cylinder of transparent gel material that will lie beneath it.

In some embodiments, the transparent material comprises hyaluronic acid (HA). HA is a biocompatible material that has been approved by the FDA as a subcutaneous injectable for cosmetic use. HA is a clear, transparent, colorless material when in the form of a gel. Therefore, it has excellent light transmission properties. Preferably, the HA is cross-linked. When it is cross-linked, HA has enhanced resistance to proteolytic degradation. HA also appears to have interesting anti-microbial properties. Zaleski, Antimicrob. Agents Chemother., 50(11) November 2006 3856-60, reports that HA-binding peptides prevent experimental staph. aureus wound infections. HA has also been used as an anti-infective coating upon implants. See US Patent Publication No. 2005-0153429. In some embodiments, the HA is Juvederm™, marketed by Allergan.

In some embodiments, the transparent gel material may be mixed with antibiotics or angiogenesis-inhibiting materials.

Therefore, in accordance with the present invention, there is provided a method comprising the steps of:
a) placing a light collecting implant in the skull,
b) removing a core of subepidermal tissue from a portion of the scalp above the light collecting implant, and
c) replacing the core with a transparent material.

Now referring to FIG. 6e, there is provided a cross-section of the patient's skull and scalp implanted with the light-collecting implant 403 and the transparent replacement material 601. External light source 603 irradiates the epidermis above the replacement material. This light will pass through the transparent epidermis and the transparent replacement material, and then enter the proximal end of the light collector. This should greatly attenuate any light loss associated with transcutaneous light delivery.

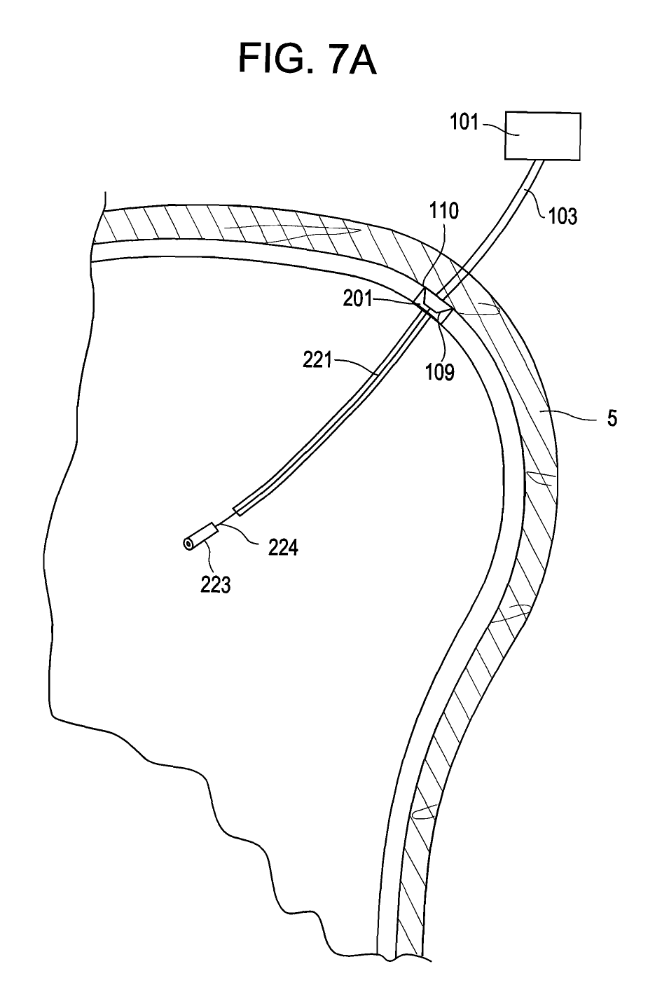

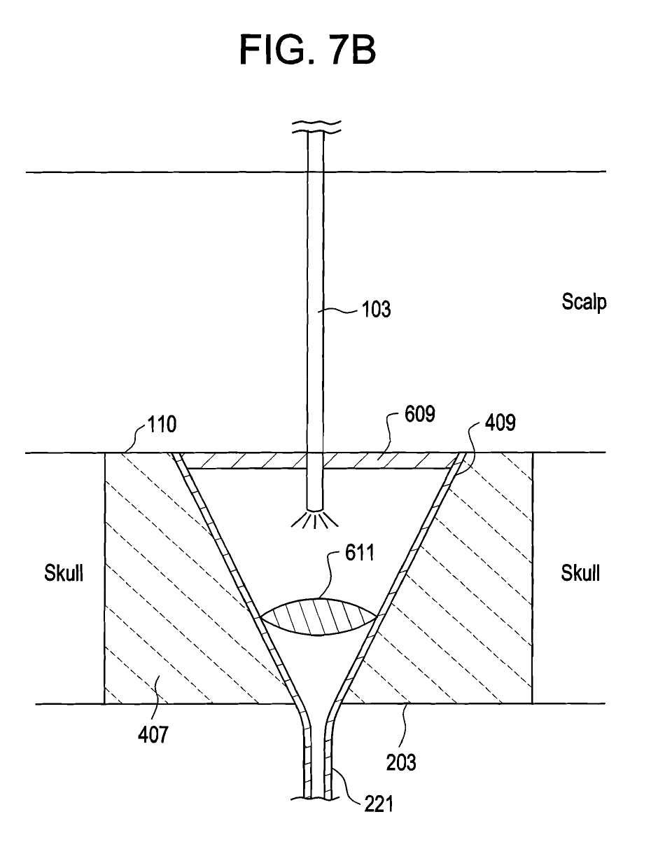

Now referring to FIG. 7a, there is provided an implant having an external light source. The externally based-control device has a light source 101 for generating light within the device. The light generated by this source is transmitted through optical wave guide 103 through the patient's skin S to an internally-based light port 109 provided on the proximal surface 110 of the implant 201. The light port is adapted to be in light-communication with optical wave guide 221 disposed upon the distal surface 203 surface of the implant. The tube 223 disposed upon the distal portion 224 of the optical wave guide receives the light and transmit the light to the adjacent brain tissue.

Now referring to FIG. 7b, in some embodiments in which an internally-based light port is provided on the proximal surface 110 of the implant 201, the light port comprises a flexible gasket 609 that is pierced by the needle-like optical wave guide 103. Because it does not rely upon delivery of light across scalp tissue, this embodiment can provide a guaranteed source of large amounts of red light while being only minimally invasive. In some embodiments, a convex lens 611 is placed between the optical wave guide 103 and the distal surface 203 of the implant in order to focus uncentered light upon the optical wave guide 221.

In some embodiments, there is provided a red LED implant whose power requirements are provided by transcutaneous Rf induction to create red light in vivo. As the transcutaneous delivery of Rf energy is highly predictable, this mode of energy delivery would result in the production of a guaranteed high and uniform level of light. Therefore, this mode of energy delivery eliminates the light loss issues associated with the transcutaneous delivery of red light. In some embodiments, the Rf powdered LED implant is located above the skull surface in order to provide a tactile locater for the user directing the Rf wand (to help a spouse or other provider accurately deliver the Rf energy).

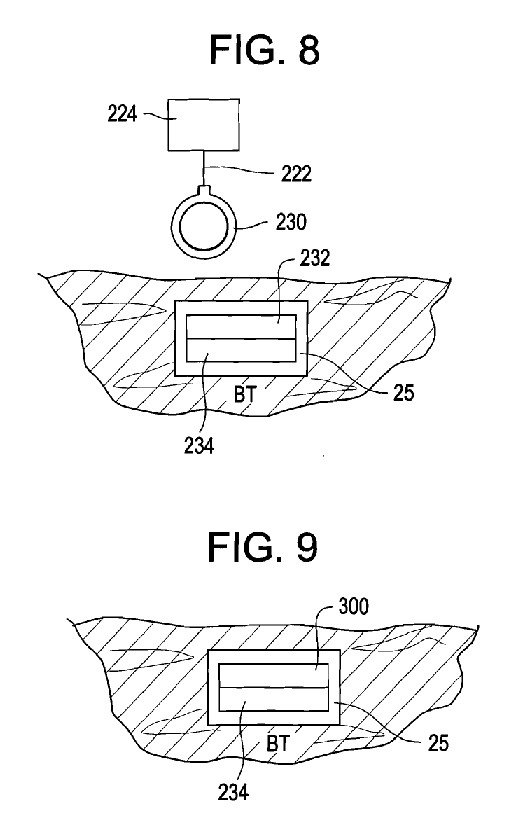

Now referring to FIG. 8, there is provided an exemplary Red light unit having an internal light source. Externally based-control device 222 has an RF energy source 224 and an antenna 230 for transmitting signals to an internally-based antenna 232 provided on the prosthesis. These antennae 230, 232 may be electro-magnetically coupled to each other. The internal antenna 232 sends electrical power to a light emitting diode (LED) 234 disposed internally on the implant in response to the transmitted signal transmitted by the external antenna 230. The light generated by the LED travels across light transparent casing 25 and into the brain tissue BT.

In some embodiments, and now referring to FIG. 9, the prosthesis having an internal light source further contains an internal power source 300, such as a battery (which could be re-chargeable), which is controlled by an internal receiver and has sufficient energy stored therein to deliver electrical power to the light source 234 in an amount sufficient to cause the desired light output.

When the implant is coupled with external energy, power can be transmitted into the internal device to re-charge the battery.

In some embodiments, the light generated by the implant is powered by wireless telemetry integrated onto or into the implant itself. In the FIG. 8 embodiment, the LED 234 may comprise a radiofrequency-to-DC converter and modulator. When radiofrequency signals are emitted by the external antenna 230 and picked up by the internal antenna 232, these signals are then converted by the receiver (not shown) into electrical current to activate the light source of the PCO unit.

In one embodiment, the implant may have an internal processor adapted to intermittently activate the LED.

In some embodiments, the telemetry portion of the device is provided by conventional, commercially-available components. For example, the externally-based power control device can be any conventional transmitter, preferably capable of transmitting at least about 40 milliwatts of energy to the internally-based antenna. Examples of such commercially available transmitters include those available from Microstrain, Inc. Burlington, Vt. Likewise, the internally-based power antenna can be any conventional antenna capable of producing at least about 40 milliwatts of energy in response to coupling with the externally-generated Rf signal. Examples of such commercially available antennae include those used in the Microstrain Strainlink™ device. Conventional transmitter-receiver telemetry is capable of transmitting up to about 500 milliwatts of energy to the internally-based antenna.

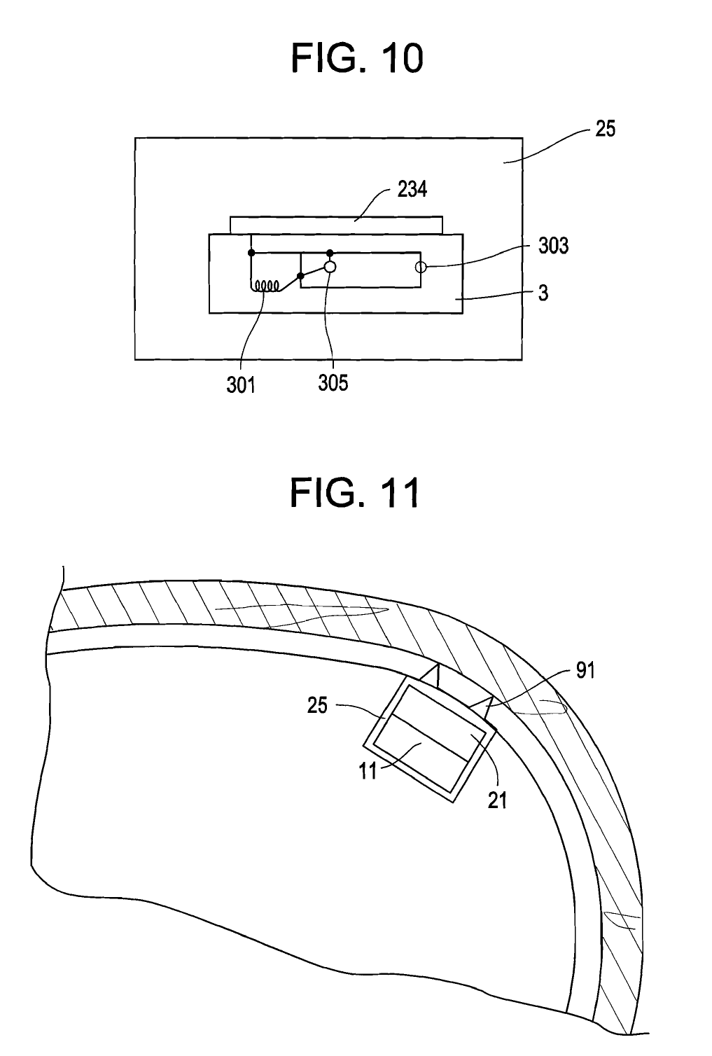

In some embodiments, and now referring to FIG. 10, the implant includes a light emitting diode (LED) 234 built upon a base portion 3 of the implant, along with the required components to achieve trans-dermal activation and powering of the device. These components can include, but are not limited to, RF coils 301, control circuitry 303, a battery 305, and a capacitor. Such a device could be capable of intermittent or sustained activation without penetrating the skin, thereby avoiding trauma to the patient and/or risk of infection from skin-borne bacteria. As shown above, the accessory items needed to power and control the LED may be embedded within the implant. However, they could also be located on the surface(s) of the implant, or at a site adjacent to or near the implant, and in communication with the implant.

In some embodiments, the light source is provided on the implant and is adapted to be permanently implanted into the patient. The advantage of the internal light source is that there is no need for further transcutaneous invasion of the patient. Rather, the internally-disposed light source is activated by either a battery disposed on the implant, or by telemetry, or both. In some embodiments of the present invention using an internal light source, the light source is provided by a bioMEMs component.

Because use of the present invention may require its repeated activation by Rf energy, it would be helpful if the user could be guaranteed that the implant remained in the same place within the skull. Accordingly, in some embodiments, and now referring to FIG. 11, the device of the present invention comprises anchors 91, preferably projecting from the casing 25. Preferably, the anchors are placed on the proximal side of the device, adjacent the antenna 21. In this position, the anchor may be inserted into the bone of the skull S, thereby insuring its position.

In some embodiments, the light source comprises a chest-implanted capacitor with a 10 year life span as the energy source thereto. In some embodiments, a red light source or red light collector and the proximal end of the optical wave guideare placed in the chest. This allows the surgeon to conduct maintenance activity on an implanted light source without having to re-open the cranium. In addition, location within the chest also lessens the chances of surface erosion.

The present invention may also be used to treat head and neck pain caused by cancer.

We claim:

1. A method of treating a patient having chronic pain, comprising the steps of:
a) providing a optical wave guide having a proximal end portion and a distal end portion having a translucent light diffuser attached thereto;
b) implanting the translucent light diffuser into the patient's cerebral aqueduct, and
c) delivering light through the optical wave guide and translucent light diffuser to irradiate at least a portion of a periaqueductal gray with an effective amount of light.

2. The method of claim 1 wherein the diameter of the translucent light diffuser is at least two times the diameter of the cerebral aqueduct.

3. The method of claim 1 wherein the diameter of the translucent light diffuser is at least three times the diameter of the cerebral aqueduct.

4. The method of claim 1 wherein the diameter of the translucent light diffuser has a tube shape.

5. The method of claim 1 wherein the length of the translucent light diffuser is at least 25% of the length of the cerebral aqueduct.

6. The method of claim 1 wherein the length of the translucent light diffuser is at least 50% of the length of the cerebral aqueduct.

7. The method of claim 1 wherein the length of the translucent light diffuser is at least 75% of the length of the cerebral aqueduct.

8. The method of claim 1 wherein the effective amount of light causes release of endorphins from the periaqueductal gray.

9. The method of claim 1 wherein the effective amount of light is delivered in an energy density of between 1 J/cm² and 10 J/cm².

10. The method of claim 1 wherein the effective amount of light is delivered in a wavelength of between 600 nm and 900 nm.

11. An intracranial light delivery system, comprising:
a) a light source,
b) an optical wave guide having a proximal end connected to the light source and a distal end, and
c) a translucent light diffuser connected to the distal end of the optical wave guide.

12. The system of claim 11 wherein the translucent light diffuser comprises silicone.

13. The system of claim 11 wherein the translucent light diffuser comprises antibiotics.

14. The system of claim 11 wherein the translucent light diffuser comprises features that increase the radial transmission of light through its outer surface.

15. The system of claim 11 wherein the translucent light diffuser comprises diffractive elements embedded within the translucent tube in order to diffraction light traveling down the length of the light diffuser to exit the light diffuser in a radial direction.

16. The system of claim 11 wherein the diffractive elements comprise metallic particles.

17. The system of claim 11 wherein the translucent light diffuser comprises an outer surface that is etched in order to diffract light that is traveling down the length of the light diffuser to exit the light diffuser in a radial direction.

18. The system of claim 11 wherein the translucent light diffuser comprises an outer surface that is coated with a reflective coating to deflect axially-traveling light back into the light diffuser.

19. The system of claim 11 wherein the translucent light diffuser comprises an inner surface that is coated with a reflective coating to deflect light back into the light diffuser.

20. The system of claim 11 wherein the translucent light diffuser comprises an outer surface that is coated with an adhesive.

21. The system of claim 11 wherein the translucent light diffuser comprises a tube shape.

22. The system of claim 11 wherein the translucent light diffuser comprises a helical shape.

23. The system of claim 11 wherein the translucent light diffuser comprises a standoff.

24. A method of treating a patient having chronic pain, comprising the steps of:
a) endoscopically implanting a translucent light diffuser into the patient's cerebral aqueduct, and
b) delivering light through the translucent light diffuser to irradiate at least a portion of a periaqueductal grey with an effective amount of light.

25. The method of claim 24 further comprising the step of:
inserting a rigid neuroendoscope into the lateral ventricle.

26. The method of claim 25 further comprising the step of:
advancing the endoscope into the third ventricle.

27. The method of claim 26 further comprising the step of:
advancing the endoscope into the cerebral aqueduct.

31. A method of treating a patient having chronic pain, comprising the steps of:
a) providing a optical wave guide having a distal end portion having a translucent light diffuser attached thereto, and
b) endoscopically implanting the translucent light diffuser in the patient's periaqueductal gray.

41. A method of treating a patient having chronic pain, comprising the steps of:
a) providing a rigid neuroendoscope holding a translucent light diffuser having a optical wave guide attached thereto, and
b) inserting the neuroendoscope into the lateral ventricle.

42. The method of claim 41 further comprising the step of:
c) advancing the endoscope into the third ventricle.

43. The method of claim 42 further comprising the step of:
d) advancing the endoscope into the cerebral aqueduct.

44. The method of claim 43 further comprising the step of:
e) implanting the translucent light diffuser in the patient's cerebral aqueduct.

45. An intracranial light delivery system, comprising:
a) an energy supply source,
b) a controlling logical module,
c) connecting wires, and
d) a potted light-emitting diode array

46. The system of claim 45 wherein the light-emitting diode array is potted in such a configuration to place the diodes in discrete locations to illuminate the desired portion of the cerebral aqueduct.

47. The system of claim 45 wherein the light-emitting diode array contains at least one photo diode to measure at least one local light energy level.

48. The system of claim 45 wherein individual potted light-emitting diodes illuminate discrete segments of the cerebral aqueduct at different times.

49. The system of claim 45 wherein the potted light-emitting diodes are arranged in such a way as to create a mechanical interference fit with the local tissue contours.

50. The system of claim 45 wherein at least a section of the potting material is partially flexible post-implantation.

51. The system of claim 45 wherein at least a section of the potting material becomes substantially rigid during the post-implantation period.

52. The system of claim 45 wherein at least a portion of the potting material is translucent

53. The system of claim 45 wherein at least a portion of the potting material serves to diffuse the photonic energy being broadcast from the embedded light-emitting diodes.
